(19) United States
(12) Patent Application Publication
(10) Pub. No.: US 2010/0120076 A1
(43) Pub. Date: May 13, 2010

(54) METHOD FOR ANTENATAL ESTIMATION OF RISK OF ANEUPLOIDY

(76) Inventors: Gur Braun, Meitar (IL); Naama Marcus-Braun, Meitar (IL); Ohad Birk, Rehovot (IL)

Correspondence Address: NIXON & VANDERHYE, PC, 901 NORTH GLEBE ROAD, 11TH FLOOR, ARLINGTON, VA, 22203, US

(21) Appl. No.: 12/448,220

(22) PCT Filed: Dec. 13, 2007
(86) PCT No.: PCT/IL2007/001547

§ 371 Date: Dec. 28, 2009

(30) Foreign Application Priority Data
Dec. 14, 2006 (IL) 180095

(51) Int. Cl. C12Q 1/02

(52) U.S. Cl. 435/29

(57) ABSTRACT

The present invention relates to a system and a method for evaluating the risk of carrying a fetus with genetic anomalies such as aneuploidy and in particular, to such a system and method where a screening system and method is provided to identify fetus' having trisomy-21 (Down's syndrome) with the use of biochemical marker concentrations evaluated from the maternal blood serum.

### FIELD OF THE INVENTION

The present invention relates to a system and a method for evaluating the risk of carrying a fetus with genetic anomalies such as aneuploidy and in particular, to such a system and method where a screening system and method is provided to identify a fetus having trisomy-21 (Down's syndrome) with the use of biochemical marker concentrations evaluated from the maternal blood serum.

### BACKGROUND OF THE INVENTION

Down's syndrome, also known as trisomy-21, is one type of aneuploidy that is caused when a fetus has three copies of chromosome 21. Similarly, trisomy 13 and 18 are also a type of aneuploidy where there is an extra copy of chromosome 13 or 18. Although about 0.5% of children are born with chromosomal anomalies, the most common of the anomalies is trisomy-21.

Aneuploidy is generally defined as having an abnormal number of chromosomes, either too many or too few chromosomes. Such conditions in human fetuses lead to abnormal fetal development that may be detected during different stages of pregnancy. An abnormal number of chromosomes may be detected in various ways. The best way to identify the chromosomal composition of a fetus is through karyotyping. Karyotyping is usually done through an amniocentesis or chorionic villus sampling. However, both methods of karyotyping are invasive and pose a threat to the fetus as the procedures may lead miscarriage. Therefore usually, karyotyping is undertaken only when there is believed to be an increased risk for having an aneuploid fetus. Prior to undertaking invasive procedures the relative risk is evaluated by various risk factors and minimally invasive tests.

One known risk factor is increased maternal age. Other risk factors may be determined by evaluating markers by minimally invasive procedures such as ultrasound, urine and/or a blood test. For example, the fetal growth rate may be determined by ultrasound where developmental milestones and sizing may be used as markers for abnormal development, including Down's syndrome. Maternal urine or blood tests are used to evaluate biochemical markers found in the maternal blood serum or urine. An increase or decrease in the concentration of the biochemical marker may indicate an increased risk for aneuploidy, such as Down's syndrome.

The biochemical markers commonly used to evaluate the risk of aneuploidy in a fetus include alpha-fetoprotein (AFP), human chorionic gonadotropin (hCG; hCG has different measurable forms including total and/or the alpha or beta subunits separately, depending upon when the analysis is performed), unconjugated estradiols (UE3), pregnancy associated protein A (PAPP-A), inhibin-A, DHEA, human placental lactogen (HPL), estrogen, progesterone and so forth.

Commonly used ultrasonographic markers include the nuchal translucency (NT) score, and various long bone and cranial measurements. Each marker may behave differently over time or in an abnormal developmental situation. Where one marker may increase due to aneuploidy, a different marker may decrease due to the same condition.

It is well documented that low maternal serum alpha-fetoprotein (AFP) is associated with Down's syndrome [1, 2]. However, considered in isolation the AFP level does not provide a sufficiently high detection rate and yields too many false-positive results that entail unnecessary invasive karyotyping procedures. Other markers that are associated with aneuploidy and in particular Down's syndrome include an increased level of maternal serum beta-human chorionic gonadotropin (hCG), low levels of maternal unconjugated estriol (UE3) [3, 4], increased levels of inhibin-A and low levels of PAPP-A. These makers are used in conjunction with other risk factors to evaluate the overall risk of fetal aneuploidy.

In order to minimize the need for the invasive karyotyping the screening tests need to maximize the detection rate of those fetuses at high risk for aneuploidy, so as to minimize further diagnostic tests with their attendant risks.

The effectiveness of a screening test depends on its ability to discriminate between pregnancies with Down's syndrome and unaffected pregnancies. The discriminatory power of a test is usually specified in terms of the detection rate achieved for a given false-positive rate, or in terms of the false-positive rate required to achieve a given detection rate. The detection rate is the proportion of Down's syndrome pregnancies with a positive result. The false-positive rate is the proportion of unaffected pregnancies where the screening tests show a positive result, or high risk of trisomy-21. Different screening markers generally impart more discriminatory power to a screening test at one stage of the pregnancy than at other stages. Currently employed screening tests rely on certain combinations of biochemical and ultrasound markers that have been identified as being effective when used together at a specific, single stage of pregnancy; yet these tests still result in many false positives and/or false negatives.

For example, the “combined test” performed in the first trimester using nuchal translucency and free [beta]-hCG and PAPP-A as screening markers can achieve an 80% detection rate with a 5% false-positive rate. The “triple test” performed in the second trimester uses AFP, UE3 and hCG as screening markers. The “quadruple test” performed in the second trimester uses the screening markers of the “triple test” plus inhibin-A. The “triple test” and “quadruple test” can achieve an 80% detection rate with a false positive rate of 10% and 6.6% respectively.

However, a screening test with greater discriminatory power would be desirable. A high false-positive rate means that a large number of women with screen-positive results in fact have unaffected pregnancies. For these unaffected women the screen-positive result, quite apart from causing considerable anxiety, might lead to a diagnostic procedure such as amniocentesis or chorionic villus sampling which has a risk of miscarriage of about 1 in 100.

U.S. Pat. No. 5,506,150 to Canick et al, U.S. Pat. No. 6,573,103 to Wald, Taiwanese Patent No. TW261516 to Jeng-Shiou and U.S. Pat. No. 5,252,489 to Macri, teach the use of biochemical and ultrasound markers in determining the antennal risk of Down's syndrome. However, all use a calculation of the multiple of mean and compare individual markers against expected levels of the individual marker. The prior art uniformly suggests the combined use of different markers but only in an additive form. Therefore the combined use of the marker provides improved results but still results in excessive false positive and false negative results.

U.S. Pat. No. 5,506,150 to Canick et al introduces a new marker, DHEAS, to be used in conjunction with the known biochemical markers, but still in an additive format.

U.S. Pat. No. 6,573,103 to Wald describes a screening test using various ultrasound and biochemical markers to evaluate the risk for individual fetuses relative to the population; however, the analysis still relates to additive results.

Taiwanese Patent No. TW261516 to Jeng-Shiou uses regression analysis to identify a second trimester screen however they too suffer from the use of an abundant number of markers including NT, beta-hCG, AFP, and maternal age.

### SUMMARY OF THE INVENTION

There is an unmet need for, and it would be highly useful to have, a system and a method for screening for aneuploidy in a fetus in a comprehensive manner that reveals a lowered false positive detection and increased detection rate.

Currently markers are combined in an individualized manner, by considering the individual multiple of mean, (MoM). Therefore the tests do not take into account the relative effect that one marker may have on another. As the evaluation of the markers is additive, a comparative process is not currently known or used.

The present invention overcomes these deficiencies of the background by providing a system and method for fetal aneuploidy detection by considering the interactive properties of at least two markers through a comparison function, for example (in some embodiments) in ratio form. The use of a comparison function enables the relative behavior of markers to be examined, by evaluating their concentration ratio, thereby providing a reliable system and method to evaluate antennal risk of aneuploidy and in particular trisomy-21, with an improved false positive ratio.

A preferred embodiment of the present invention provides for a method to account for the concerted changes between at least two or more markers according to a comparison function. Although the individual use of markers and different additive combination thereof has shown promising results, they do not indicate the relationship between markers and in particular they do not indicate the relationship between marker levels during fetal development, and therefore they do not provide a complete picture. That is, although it is known in the art that aneuploid fetuses exhibit reduced concentrations of AFP, UE3 and PAPP-A, while beta-hCG and inhibin-A exhibit increased concentrations, their concerted effects have not been reported in the art.

A preferred embodiment of the present invention provides for a method, system, kit and apparatus for evaluating the risk of fetal aneuploidy by determining a ratio of the biochemical markers.

A preferred embodiment of the present invention provides for an improved system and method for fetal aneuploidy screening by obtaining at least one or more ratio of biochemical markers associated with increased risk of aneuploidy and determining a ratio of their relevant concentrations. Optionally, the ratio may be determined from at least two marker concentrations for example including but not limited to AFP, HCG, UE3, PAPP-A, inhibin-A, DHEA, HPL, estrogen, progesterone, or the like, producing a ratio for example including but not limited to [AFP]/[hCG], [UE3]/[hCG], [HPL]/[HCG], [estrone]/[HCG], [Estradiol]/[HCG]; [Progesterone]/[HCG] or the like. Optionally, the ratio is composed of two concentrations of biomarkers wherein the ratio's numerator comprises a biomarker who's concentration is reduced in aneuploid fetuses while the denominator comprises a biomarker concentration who's concentration levels are increased in aneuploid fetuses.

Optionally, ultrasound markers, maternal medical history data and/or other data may be used to evaluate the overall risk of fetal aneuploidy.

A preferred embodiment of the present invention uses the ratio of [AFP]/[hCG] referred to as phi (φ) that undergoes statistical analysis to determine the relative probability or risk for fetal aneuploidy. The method according to a preferred embodiment of the present invention preferably comprises the following stages: determine fetal gestation age optionally by last menstrual period (LMP) or ultrasound (US); obtain a maternal sample, preferably a fluid sample, optionally a urine sample or most preferably a blood sample; analyze the sample for at least two or more biochemical markers; and determine the ratio of concentrations found in the sample. Optionally the method further comprises the following stages: obtain and account for additional non biochemical data; for example including maternal weight, age, familial history or the like; and perform statistical analysis preferably by compare subject results to the normal distribution, negative results, and aneuploid distribution, positive results, to obtain the probability.

An additional preferred embodiment of the present invention uses the ratio of two biochemical markers to further evaluate and analyze successive screening results to reduce the false positive rate of the aneuploidy screening test according to the present invention. According to this preferred embodiment the rate of change of the ratio over time is used to further reduce the false positive results and preferably also the false negative results.

A comparison of the rate of change of the concentration ratio between normal and trisomy-21 reveals that the ratio rises faster in a normal fetus than it does in an aneuploid fetus. The curve of the rate of change may optionally be determined by any number of mathematical techniques including linear or nonlinear analysis, for example including but not limited to exponential, polynomial analysis, high order polynomial, power, moving average, logarithmic, or the like.

Preferably, determining the rate of change between two samples comprises the following stages: determine fetal gestational age optionally by last menstrual period (LMP) or ultrasound (US) at the time the tests were taken; and obtain a first fetal aneuploidy probability ratio, preferably including but not limited to AFP/HCG. Optionally this may be provided or optionally calculated based on raw results from a first test.

Next, a second fetal aneuploidy probability ratio is obtained according to a preferred embodiment of the present invention, preferably including but not limited to AFP/HCG. Statistical analysis is performed, preferably by comparing subject results to the normal distribution, negative results, and aneuploid distribution, positive results, to obtain the probability.

Preferably, there is a time delay of about 1 week between first ratio assessment and a second ratio assessment. Preferably, both ratio readings are used to depict the rate of change of the ratio over the time delay (1 week). Optionally a longer time delay may be used. Preferably, the obtained rate of change is compared to a normal population distribution and an aneuploid population distribution.

Preferably the rate of change of the ratio risk factor is optionally determined mathematically to solve for the rate of change of the ratio over time, R_{P}=(δφ/δτ) where R is the rate of change of the ratio, (δφ) depicts the difference in the obtained ratio, over time δτ. If the obtained rate is above a threshold and approaches the normal population R_{P}, the pregnancy is defined to be normal. Optionally, in cases where the Rₚ obtained is lower and stands below a certain threshold, preferably determined for individual ethnic groups, such a low rate identifies pregnancies at a high risk for a high chance of carrying a Down's syndrome fetus.

A further optional embodiment of the present invention provides for the optional use of a slope curve greater or equal to a cut-off slope, which is greater than the slope of the Down syndrome pregnancies:

{(b₁a₂−b₂a₁)/[b₂b₁(τ₂−τ₁)]}ₙ≧{(b₁a₂−b₂a₁)/[b₂b₁(τ₂−τ₁)]}_{d}*k

Where b₁ and b₂ are two concentrations of β-hCG obtained respectively at gestational ages τ₂ and at τ₁; a₁ and a₂ are the corresponding alpha-fetoprotein (AFP) concentrations, while n stands for normal pregnancies, which are suspected to be false positives; and d stands for known Down's syndrome pregnancies. Optionally, k is used to determine the false positive threshold cutoff curve. The threshold curve may be derived from the Down's syndrome distribution curve by factoring in k. The threshold curve determined via k is used to minimize the false positive rate while maximizing the detection rate. As the value of k increases, the number of false positive cases estimated as Down syndrome cases also increases. Optionally, the system according to an optional embodiment of the present invention provides for automatic determination of k. Optionally, the system according the present invention determines the aneuploidy distribution curve and the normal distribution curve, from the available data, therefore is able to determine k required to achieve a predetermined false positive rate. Optionally, the false positive rate is determined by a user to maximize the true detection rate while minimizing the false positive rate.

For example, the false positive rate may be kept to 5% while providing close to 100% reliability with a low k value, estimated at k=1.30 for a specific ethnic group. However, the value of k must be determined for individual ethnic groups based on the respective distribution curve of the normal and aneuploid cases.

An optional embodiment of the present invention is the use of the rate of change of the ratio, to reduce the false positive rate by determining two concentration ratios phi (φ) and comparing their change over time. Normal pregnancies may often exhibit a low rate of decrease in serum beta-hCG; a low rate of increase in serum AFP; or both. In such cases, φ exhibits relatively lower levels. Therefore, some normal pregnancies, may exhibit low levels of φ at a particular gestational week, may therefore be incorrectly classified as being at high risk for aneuploidy. Effectively such cases are potentially false positive cases. However, when considering a plurality of ratios over a period of about 1 week, the rate of change of φ may reveal that the ratio's rate of change R approaches the rate of normal development. Alternatively, determining the ratio's rate of change, R, may indicate that the ratio is in fact approaching the Down's syndrome's rate of development, which may then indicate further potentially invasive testing.

The use of the ratio's rate of change to further reduce false positive rate is due to the observations that the Rₚ, in sera of most normal population is higher than the rate of change of the .Down's syndrome distribution where rate (δφ_{d}/δτ)=Rd remains low.

Preferably, the risk analysis according to the system and method of any of the embodiments of the present invention is performed during the second trimester, during the time of gestational age 14-24 weeks. Most preferably the screening test according to the present invention is performed between weeks 16-20. Optionally, the analysis may be performed during the first trimester, gestational age 0-13 weeks. Optionally, the analysis may be performed intermittently prior to the third trimester.

In any of the embodiments of the present invention, the biochemical marker concentrations may optionally be determined by any technique or method known and accepted in the art. Optionally, the relative concentrations may be determined from a noninvasive source of bodily fluid for example including but not limited to blood or urine. Optionally biochemical marker concentrations are determined by methods including but not limited to immunoassays, binding assays, chromatography, biological activity assay and mass spectrum, fluorescence, chemiluminescence, light absorption, light scatter, color detection, or the like. The biochemical marker concentrations used to determine the ratios (φ) are preferably expressed in similar units for example including but not limited to nanograms per milliliter (ng/ml), International Units per milliliter (IU/ml) or the like.

In any of the embodiments of the present invention statistical analysis is preferably used to determine the risk of fetal aneuploidy, particularly trisomy-21. The statistical analysis for determining fetal aneuploidy probability optionally includes but is not limited to linear models, non-linear models, regression models, partial least squares models, NIPALS algorithm (PCA/PLS), non-linear estimations, polynomial estimations, exponential to estimations, fixed non-linear regressions, log-linear analyses, log-non-linear analyses, time series/forecasting structural equation modeling, survival analyses, multivariate analyses, regression analysis, logistic regression analysis, odds ratio analysis or the like that is known and accepted in the art.

Most preferably, a logistic regression analysis is performed on the ratio phi (φ) that may optionally take the form

$$p = \frac{exp ( a + b * ϕ + c * τ )}{1 + exp ( a + b * ϕ + c * τ )}$$

where φ is the concentration ratio; τ is the gestation age of the fetus at the time of the sample is tested; a, b, and c are parameters calculable from the distribution curves that depict the characteristic of any population on which the model is applied.

In any of the embodiments of the present invention, information and/or additional data or the like may be factored into the statistical analysis. For example, additional data optionally includes but is not limited to one or more of maternal age, maternal weight, maternal BMI, maternal obstetric history, familial history, familial ethnicity, or the like.

Optionally, additional data may also include (additionally or alternative) measurements of one or more other markers relating to the current gestation. For example, results of ultrasound markers such as Nuchal Translucency (NT) results, long bone measurements, fetal size measurements growth curve data determined by ultrasound, or the like may optionally be incorporated.

Optionally, additional data obtained from earlier probability calculation may also be incorporated in the screening process, additionally or alternatively. For example, the results of the triple test, integrated test, first trimester test results, second trimester test results, or the like, may optionally be incorporated.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “computer” on a “computer network”, it should be noted that optionally any device featuring a data processor and/or the ability to execute one or more instructions may be described as a computer, including but not limited to a PC (personal computer), a server, a minicomputer, a cellular telephone, a smart phone, a PDA (personal data assistant), a pager. Any two or more of such devices in communication with each other, and/or any computer in communication with any other computer, may optionally comprise a “computer network”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic block diagram of an exemplary system according to the present invention;

FIG. 2 is an exemplary method according to the present invention;

FIG. 3 is an exemplary method according to the present invention;

FIG. 4 depicts the mean [AFP]/[hCG] ratio as a function of the gestational age in normal and in trisomy-21 pregnancy. The gray areas represent the 99% confidence interval [CI] distribution of every point on the respective curves;

FIG. 5 depicts the difference {[AFP/[hCG]_{N}−[AFP]/[hCG]_{D}} between the mean [AFP]/[hCG] ratio for normal pregnancies (denoted N) and the mean ratio for trisomy-21 pregnancies (denoted D) as a function of gestational age; and

FIG. 6 is a graph that depicts the change of [AFP]/[hCG] ratio over weeks 16-20 for Trisomy-21, for false positive and normal subjects.

### DESCRIPTION OF THE PREFERRED EMBODIMENTS

The present invention is of a system and a method for an improved screening test for fetal aneuploidy and in particular Down's syndrome. The improved screening is made possible by considering and evaluating a comparison function, such as a ratio, of the biochemical markers associated with aneuploidy and specifically Down's syndrome.

The principles and operation of the present invention may be better understood with reference to the drawings and the accompanying description.

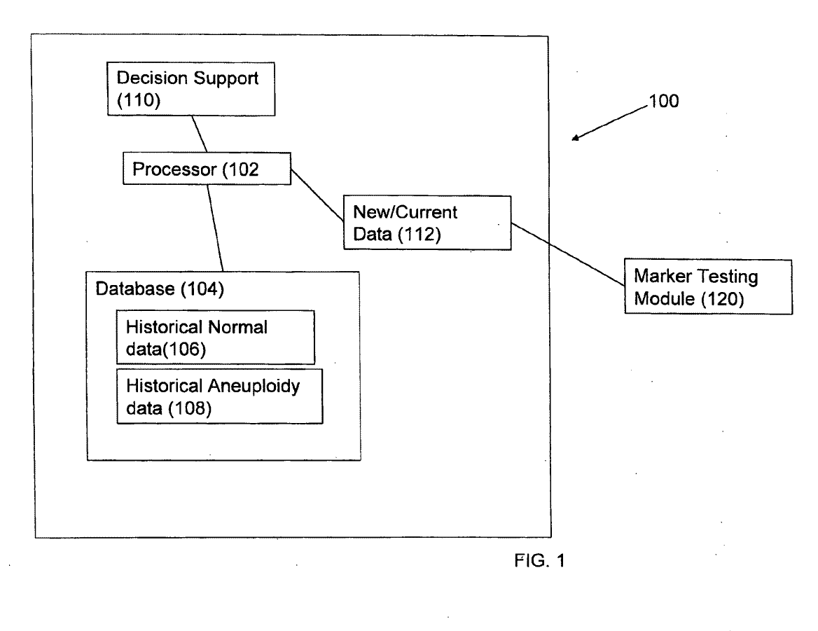

Referring now to the drawings, FIG. 1 is a schematic block diagram of an exemplary system according to the present invention for an improved screening test for Down's syndrome. FIG. 1 shows a system 100 according to the present invention comprising a marker testing module 120, processor 102, database 104, data entry interface 112, and decision support module 110. Preferably, marker testing module 120 allows the determination of the concentration of the various markers for example including but not limited to a plurality of biochemical markers and/or ultrasound markers. Optionally, ultrasound markers include one or more of nuchal translucency and fetal growth parameters, while biochemical markers include one or more of evaluation of serum levels of HCG, AFP, UE3, Inhibin-A, PAPP-A or the like. Marker testing module 120 may evaluate the chosen marker by any means accepted and known in the art, optionally including but not limited to one or more of ultrasound, immunoassays, binding assays, chromatography, biological activity assay and mass spectrum, fluorescence, chemiluminescence's, light absorption, light scatter, color detection, or the like. Optionally, marker testing module 120 provides system 100 with the raw biochemical marker concentrations for evaluation.

Biochemical marker concentrations, maternal data, optionally including but not limited to one or more of ethnicity, maternal age, gestational age, maternal weight, or maternal BMI are entered into system 100 through data entry interface 112. Preferably, newly measured concentration data is then entered through data entry interface 112 (and/or directly or automatically from marker testing module 120) which in then relayed to processor 102 to evaluate the ratio of at least two biochemical markers, preferably including but not limited to AFP and beta-HCG. Processor 102 then processes the raw concentration to obtain the ratio preferably [AFP]/[HCG]. Optionally, further data is processed for example including but not limited to one or more of the maternal data, ethnicity data, various factors and the like.

Processor 102 then retrieves information from database 104 to build the relevant distribution curves based on the maternal data, ethnicity and the like. Distribution data is stored in database 104 in historical normal data module 106 that optionally comprises data relating to gestational data, optionally from various sources including but not limited to literature data, database or the like. Similarly, aneuploid historical data module 108 is comprises historical data regarding aneuploidy cases, particularly including Down's syndrome, that allow for the comparison function result(s) to be examined with regard to normal vs. aneuploid conditions. The historical data from database 104 enables processor 102 to abstract a distribution curve respectively for normal fetal development and for aneuploid development.

Optionally and preferably processor 102 evaluates the ratio distribution curve over the relevant time frame, preferably the second trimester. Once the distribution curves have been abstracted by processor 102, the curves are compared to the new data is compared to determine the risk of fetal aneuploidy. The comparative results are then preferably further processed in the decision support module 110 to produce a risk factor and optionally a mode of action relative to the risk factor, including but not limited to suggested mode of action or treatment, to perform a second screening test or to perform amniocentesis and the like.

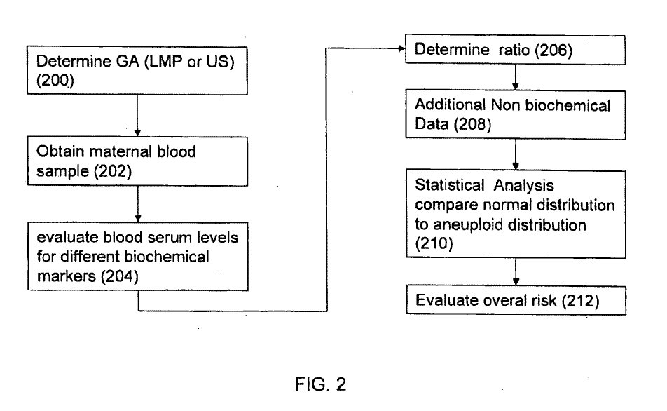

FIG. 2 shows a flowchart of an exemplary method according to some embodiments of the present invention for performing the comparison function according to the ratio. In stage 200 the gestational age of the fetal subject is determined optionally by ultrasound, last menstrual period, or the like. Preferably, a maternal blood sample is obtained in stage 202, to determine the relative concentration levels of a plurality of biochemical markers in the blood serum. Blood serum levels are evaluated during stage 204 optionally by any means known and accepted in the art. Stage 204 reveals the relative concentrations in the maternal blood serum that are used to determine the ratio in stage 206, as a non-limiting example of a comparison function according to the present invention. Preferably the ratio is of the levels of the markers AFP and HCG, according to AFP/HCG, to evaluate the antennal risk of aneuploidy and particularly Down's syndrome. Next in stage 208 additional non biochemical data is optionally and preferably evaluated for example including but not limited to ethnicity, maternal age, or the like. In stage 210 the statistical analysis is preferably performed, more preferably through a logistic regression analyses. More is preferably a distribution curve is obtained for both the aneuploid population and the normal population based on historical data, according to gestational age, and ethnicity, maternal age or like parameters. Once the statistical analysis is completed the risk or probability of aneuploidy is determined in stage 212.

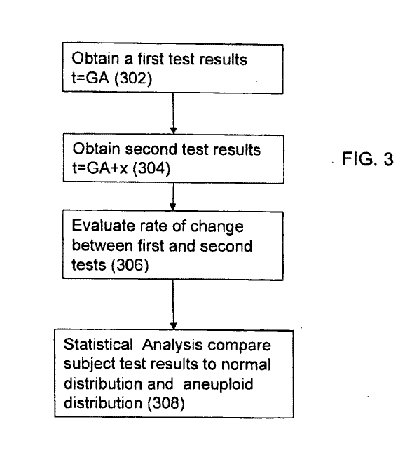

FIG. 3 depicts a method according to an optional embodiment of the present invention wherein the comparison function is determined according to the rate of change of the ratio of a plurality of biochemical markers, preferably obtained as described with regard to FIG. 2. The result of the comparison function is evaluated to determine the risk for trisomy-21, for example to reduce the number of false positive scores. Reducing the false positive score is paramount as it saves the fetus from risk due to invasive procedures. Most preferably, for the method described below, the ratio of biochemical markers is obtained during two separate occasions, more preferably determined at least 1 week apart. As for FIG. 2, the preferred ratio comprises AFP/HCG.

In stage 302 the first test result data is obtained relative to an earlier gestational age that is preferably more than 15 weeks and less then 20 weeks. In stage 304 the parallel test results are obtained from another test, preferably performed at least one week after those of stage 302. In stage 306 the rate of change is evaluated, based on the different test results and the elapsed time between the initial and the secondary test results.

In stage 308 the abstracted statistical data is preferably compared to rate distribution charts based on historical data that is relative to ethnicity, the initial ratio to determine if the test subject is at a high risk for Down's syndrome or other aneuploid condition, based on the proximity and relationship to the distribution curve.

### Example 1

### [AFP]/[HCG] Ratio

The below Example relates to actual experimental data, obtained as described, as a non-limiting, illustrative description of an optional method for performing the present invention according to some embodiments.

Trisomy-21 cases, which were detected through karyotyping at the Genetics Institute of Soroka University Medical Center (Beer Sheva, Israel) over a period of 15 years (between January 1987 and April 2002), were analyzed retrospectively. Altogether, 113 cases of trisomy-21 were detected throughout that period, of which 51 cases could be included in the present set of data. Some of these cases were diagnosed during pregnancy by amniocentesis or by chorionic-villus sampling, while others were detected only after birth. The total number of normal pregnancies, randomly selected to form a control group, was 10365.

Data collected for all pregnancies included the absolute concentrations of AFP in IU/ml and of beta-hCG in IU/ml. Women's age and gestational age were documented. Gestational age was determined by first trimester ultrasound scan (US) or by last menstrual period (LMP). Serum levels of AFP and beta-hCG had been determined by standard immunoassays. In 53 cases of the above 113 cases, absolute concentrations of AFP and beta-hCG were available, and of those 53 cases, 51 were included in the statistical analyses. Two subjects were excluded because gestational age was not identified. The AFP and beta-hCG population means of the normal pregnancies tested during these years were used as reference values for each respective gestational week. Gestational ages, included in the study, comprise weeks 15 to 20.

The distribution of the number of trisomy-21 cases diagnosed at different gestational ages is presented in Table 1, together with normal pregnancies at the various gestational ages. The table includes the mean [AFP]/[hCG] ratios in normal and trisomy-21 pregnancies at each gestational week, and the corresponding standard deviations (sd) that were calculated, as well as P-values, according to the sign test. As seen in Table 1, the [AFP]/[hCG] ratio was significantly different for trisomy-21 and for normal pregnancies, especially at gestational weeks 16 to 19, where the [AFP]/[hCG] ratio is clearly shown to be significantly lower in pregnancies having trisomy-21 fetuses when compared to the normal group.

TABLE 1
AFP/hCG ratios obtained during normal and trisomy-21 pregnancies
at gestational age ranging from 16 to 19 weeks.

| Gestational | Normal | | | Trisomy-21 | | | |
|---|---|---|---|---|---|---|---|
| Age | N | mean | SD | N | mean | SD | P-value |
| | | 1.10 | | | | | |
| 16 | 2861 | 1.52 | 0.01 | 17 | 0.41 | 0.34 | 0.0003 |
| 17 | 3925 | 2.07 | 0.02 | 12 | 0.88 | 0.50 | 0.0005 |
| 18 | 1928 | 2.66 | 0.04 | 9 | 0.69 | 0.44 | 0.0039 |
| 19 | 1036 | 3.57 | 0.03 | 8 | 1.34 | 0.63 | 0.0078 |
| | | 4.19 | | | | | |

N column represents the sample size, mean value, SD represents the standard deviation of the sample, P-value depicts the results of the sign test where P < 0.50 indicates mean [AFP]/[hCG] ratio values are significantly lower in trisomy-21 fetuses.

Table 2 presents the 99% confidence interval (CI) obtained at each gestational week for normal and trisomy-21 pregnancies. No overlap can be observed between these intervals, and the gap between normal and trisomy-21 cases increases with gestational age.

TABLE 2
Confidence Interval 99% for Normal and Trisomy-21 pregnancies.

| Gestational Week | Normal | Trisomy-21 |
|---|---|---|
| 15 | (1.09; 1.12) | (0.18; 0.74) |
| 16 | (1.52; 1.53) | (0.30; 0.60) |
| 17 | (2.05; 2.07) | (0.45; 1.60) |
| 18 | (2.63; 2.67) | (0.27; 1.62) |
| 19 | (3.56; 3.56) | (0.33; 2.18) |
| 20 | (4.07; 4.31) | (0.65; 1.79) |

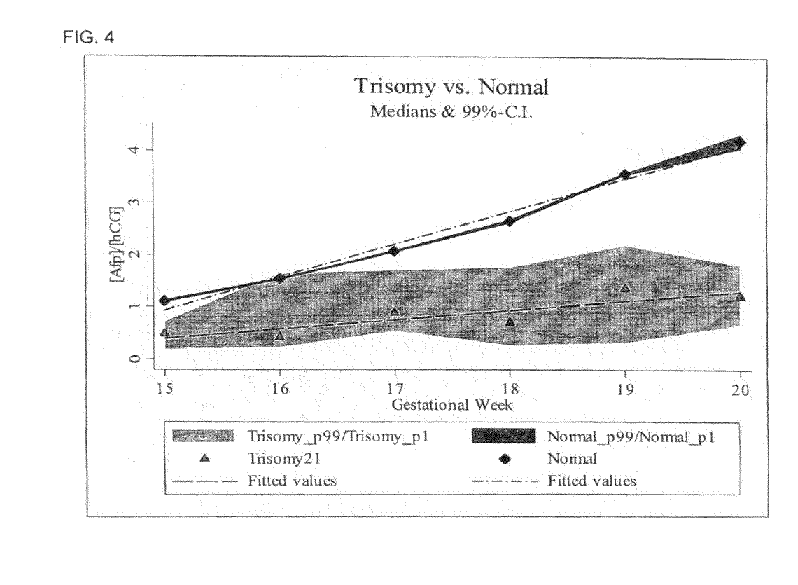

FIG. 4 graphically depicts the results of linear regression analysis on the data represented in Table 1 and Table 2. FIG. 4 shows the 99% confidence interval, in the shaded areas respectively for each of the normal cases (dark grey shading) and the trisomy-21 cases (light grey shading). Also the fitted curves clearly show that the [AFP]/[hCG] ratio for pregnancies having trisomy-21 is significantly and continuously lower than normal cases, between weeks 15-20, as indicated in Table 1, using the sign test.

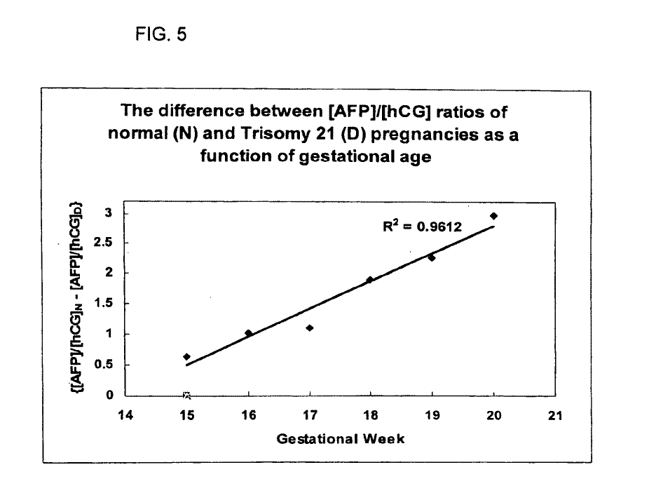

The data of Tables 1 and 2 was also used to calculate the difference {[AFP/[hCG]_{N}−[AFP]/[hCG]_{D}} between the mean [AFP]/[hCG] ratio for normal pregnancies (denoted N) and the mean ratio for trisomy-21 pregnancies (denoted D) as a function of gestational age. The results, plotted in FIG. 5, show a linear increase in the difference between the groups, between weeks 15-20.

FIG. 4 indicates that a single point ratio measurement between weeks 15 and 20 may be used as a screening test for trisomy-21, in a reliable manner according to a preferred embodiment of the present invention. Similarly, FIG. 5 shows that a plurality of ratio measurements may also serve a good basis to determine the likelihood of trisomy-21, as over time the ratios continuously diverge.

### Example 2

### Comparative Linear Regression Slope

The below Example relates to actual experimental data, obtained as described, as a non-limiting, illustrative description of an optional method for performing the present invention according to some embodiments.

A multi-center study and statistical analysis was preformed to tests the value of using the biochemical marker ratio according to the present invention. The present non-limiting example depicts the results with [AFP]/[hCG] ratio, however any biomarker ratio may be used according to the present invention. The ratio tested was [AFP]/[hCG] to function as a screening test for aneuploidy, particularly trisomy-21. A linear regression model was used to test the relation between the gestational age (GA), expressed in weeks, and the ratio [AFP]/[hCG]

The abstracted regression model was applied to three individual study centers. Table 3 depicts the regression coefficients beta (β), its standard error, and the 95% confidence interval (CI) with the different study groups. Accordingly, the same regression model was applied to the different centers, false positive cases, and normal cases.

TABLE 3
Linear regression of a multicenter study,
showing the 95% confidence interval.

| Study Group | β (Slope) | Standard Error | C.I. (95%) |
|---|---|---|---|
| Center 1 | 0.284 | 0.005 | (2.74, 2.94) |
| Center 2 | 0.157 | 0.033 | (0.10, 0.22) |
| Center 3 | 0.263 | 0.031 | (0.19, 0.32) |
| False Positive Cases | 0.311 | 0.020 | (0.29, 0.33) |
| Normal Cases | 0.684 | 0.005 | (0.58, 0.78) |

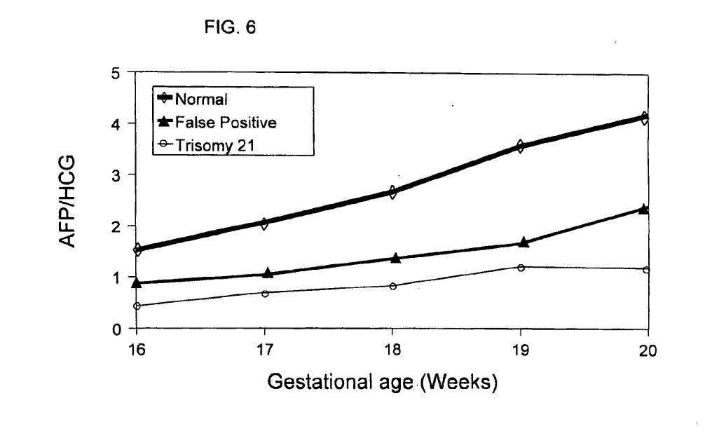

All data was then combined into three groups, namely pregnancies that were determined to be trisomy-21, false positive for trisomy-21, and normal. The abstracted regression model was then applied to the three groups whose results are presented in Table 4 and graphically in FIG. 6.

TABLE 4
Combined results

| Study Group | β (Slope) | Standard Error | C.I. (95%) |
|---|---|---|---|
| Trisomy-21 Cases | 0.238 | 0.023 | (0.19, 0.26) |
| False Positive Cases | 0.311 | 0.020 | (0.29, 0.33) |
| Normal Cases | 0.684 | 0.005 | (0.58, 0.78) |

FIG. 6 graphically depicts the results of Table 4, showing that the [AFP]/[hCG] ratio differs for the three different groups: trisomy-21 (circles), false positive (dark triangles), and normal cases (diamond) show different behavior during gestational weeks 16-20. The false positive group is of particular interest as the number of fetuses previously identified at high risk for aneuploidy, using prior art screening methods, may be significantly reduced by using the ratio according to a preferred embodiment of the present invention. The false positive curve (dark triangles) is separable and clearly identifiable from the trisomy-21 group's curve (circles) and behaves more like the normal curve (diamonds) particularly between weeks 19 and 20. During week 19 the ratio according to the present invention is increasing in both false positive (dark triangles) and normal (diamonds) groups while the Trisomy-21 group's curve (circles) plateaus.

The system according to an optional embodiment of the present invention optionally may determine the best false positive threshold curve (dark triangles) based on the distribution of Trisomy-21 cases (circles) and Normal cases (diamonds). Optionally, the accepted false positive rate is optionally entered by a user where the system determines the k value required to obtain the chosen false positive rate from the Down's syndrome distribution and the normal distribution.

This further indicates that the screening system and method according to the present invention provides a screening tool that offers increased resolution and accuracy for preventing a false positive diagnosis, than that offered by current aneuploidy screening tests, for example including the triple test. Accordingly, the ratio according to a preferred embodiment of the present invention may significantly reduce the false positive rate of current aneuploidy screening methods.

### Example 3

### False positive analysis ratio vs. triple Test

The below Example relates to actual experimental data, obtained as described, as a non-limiting, illustrative description of an optional method for performing the present invention according to some embodiments.

Example 3 depicts further analysis that was performed on the false positive group described in Example 2. Particularly, the analysis was preformed on false positive results where at least a 1:380 risk evaluation was obtained with the triple test, a commonly used screening tool, at various gestational ages during the second trimester. The false positive data was split into three groups relative to the risk assessment obtained with the triple test. The three groups were defined as follows: 1:250 to 1:380 (345 cases); 1:150 to 1:250 (268 cases) and 1:1 to 1:150 (450 cases).

The three groups were examined and compared to the regression model derived in Example 2. Specifically, the [AFP]/[hCG] ratio, according to a preferred embodiment of the present invention, was determined for each of the triple test's false positive results.

A comparative analysis was undertaken by examining results obtained with the triple test versus results that would have been obtained with the [AFP]/[hCG] ratio according to a preferred embodiment of the present invention, relative to the false positive threshold curve presented in FIG. 6 (dark triangles). Results below the threshold curve were determined to be a positive test, therefore the triple tests and the ratio test according to the present invention would yield the same results. Conversely, results above the threshold curve were determined to be negative according to the ratio test of the present invention and therefore different from the triple test results. Analysis was undertaken relative to the gestation age, between weeks 15 and 20; the results are presented in Table 5 below.

TABLE 5
[AFP]/[HCG] vs. Triple Test

| | AFP/beta | Percent of | Percent of | Percent of |
|---|---|---|---|---|
| | HCG | Normal cases | Normal Cases | Normal Cases |
| Gestation | Threshold | a Determined | Determined | Determined |
| Age (by | from | at Risk | at Risk | at Risk |
| week) | FIG. 6 | 1:250-1:380 | 1:150-1:250 | 1:1-1:150 |
| 16 | 0.85 | 52.3% | 39.7% | 18.3% |
| 17 | 1.0 | 52.7% | 47.7% | 20.1% |
| 18 | 1.3 | 56.9% | 39.7% | 24.4% |
| 19 | 1.5 | 45.5% | 40% | 27.7% |
| 20 | 2 | 68.8% | 42% | 30.8% |

| | | | |
|---|---|---|---|
| TOTAL | 52.2% | 42.5% | 22% |

Table 5 depicts the percent of normal cases identified using the [AFP]/[hCG] ratio according to the present invention, above the false positive threshold curve, dark triangles shown in FIG. 6, within each false positive risk group. Table 5 indicates that an average of 22% of false positive cases was found in the very high risk group, defined as having a risk of 1:1 to 1:150 for trisomy-21. Similarly, an average of 42.5% of the false positive tests in the 1:150 to 1:250 risk group would have been avoided by using the [AFP]/[hCG] ratio according to a preferred embodiment of the present invention. Furthermore, 52.2% of false positive results would have been avoided in the 1:250 to 1:380 risk groups. Table 5 indicates that from the 1063 false positive cases using the triple test, 390 or 36.7% needless, risky, karyotyping procedures could have been avoided with the use of the screening test according to the present invention. Therefore, the trisomy-21 screening test according to a preferred embodiment of the present invention would reduce the false positive rate by about 36.7% when compared to the triple test.

Moreover, it is expected that with a second screening test according to the present invention, the use of the ratio's rate of change according to the present invention would further reduce the false positive rate.

### REFERENCES

1. Merkatz I R, Nitowsky H M, Macri J N, Johnson W E. An association between low maternal serum alpha-fetoprotein and fetal chromosomal abnormalities. Am J Obstet Gynecol 148 (1984) 886-94.
2. Cuckle H S, Wald N J, Lindenbaum R H. Maternal serum alpha-fetoprotein measurement: a screening test for Down's syndrome. Lancet 28 (1984) 926-9.
3. Bogart M H, Pandian M R, Jones O W. Abnormal maternal serum chorionic gonadotropin levels in pregnancies with fetal chromosome abnormalities. Prenat Diagn 7 (1987) 623-30.
4. Canick J A, Knight G J, Palomaki G E, Haddow J E, Cuckle H S, Wald N J. Low second trimester maternal serum unconjugated oestriol in pregnancies with Down's syndrome. Br J Obstet Gynaecol 95 (1988) 330-3.
5. Wald N J, Cuckle H S, Densem J W, et al. Maternal serum screening for Down's syndrome in early pregnancy. BMJ 297 (1988) 883-7.
6. MacDonald M L, Wagner R M, Slotnick R N. Sensitivity and specificity of screening for Down's syndrome with alpha-fetoprotein, hCG, unconjugated estriol, and maternal age. Obstet Gynecol 77 (1991) 63-8.
7. Macri I N. Down syndrome screening method using dried blood samples. U.S. Pat. No. 5,252,489 (1993).
8. Wald N J. Antenatal screening for down's syndrome. U.S. Pat. No. 6,573,103 (2003).

While the invention has been described with respect to a limited number of embodiments, it will be appreciated that many variations, modifications and other applications of the invention may be made.

1.-27. (canceled)

28. A method for evaluating the risk of fetal aneuploidy, the method comprising: obtaining a biological sample from a pregnant subject; determining at least two concentrations of a biomarker from said sample; evaluating a ratio between the concentrations; and obtaining a probability of the risk from said ratio by comparing to a population distribution.

29. The method of claim 28, wherein the numerator of said ratio is chosen from a biomarker whose concentration is lower than the norm.

30. The method of claim 29 wherein said biomarker is chosen from the group consisting of alpha-fetoprotein (AFP), unconjugated oestriol (UE3), human placental lactogen (HPL), estrone, estradiol, progesterone, Pregnancy-Associated Plasma Protein A (PAPP-A).

31. The method of claim 28, wherein the denominator of said ratio is chosen from a biomarker whose concentration is higher than the norm.

32. The method of claim 28 wherein said biomarker is chosen from the group consisting of human chorionic gonadotropin (hCG), inhibin-A, free beta-hCG (fb-hCG).

33. The method of claim 28, wherein said ratio is compared to population distribution for normal and aneuploid pregnancies.

34. The method of claim 28, wherein said ratio is of AFP/fb-hCG or PAPPVfb-hCG.

35. The method of claim 28, wherein said ratio is measured at least once during the first or second trimester of the pregnancy.

36. The method of claim 35, wherein said ratio is measured at least once during weeks 8-22 of the pregnancy.

37. The method of claim 28, further comprising measuring at least one other pregnancy related parameter.

38. The method of claim 28, wherein said ratio is determined for a plurality of time points, wherein a rate of change of said ratio is determined for determining said risk.

39. The method of claim 38, wherein said numerator decreases relative to normal pregnancies and said denominator increases relative to normal pregnancies for said ratio at said plurality of time points.

40. The method of claim 38, further comprising determining whether said ratio is outside of a false positive result area or curve.

41. The method of claim 38, wherein a distribution of said rate of change is compared for normal and aneuploid pregnancies.

42. The method claim 28, wherein said aneuploid syndrome is Down's syndrome.

43. A method according to claim 28, comprising obtaining the alpha-fetoprotein concentration ([AFP]) and the free beta-human chorionic gonadotropin concentration ([fb-hCG]) in a blood sample obtained from the pregnant subject; and processing said concentrations to form a [AFP]/[fb-hCG] ratio; wherein said ratio is used to evaluate the risk of carrying a fetus with trisomy-21.

44. A method according to claim 43, wherein said obtaining comprises measuring [AFP] and [fb-hCG], or calculating said ratio, or extracting said ratio from published data.

45. A method according to claim 43, wherein said processing comprises providing a relation between said risk and said [AFP]/[fb-hCG] ratio.

46. A method for evaluating the rate of change of a biochemical marker, comprising obtaining a plurality of measurements for the marker for at least two separated time points, wherein the marker is related to fetal condition or development; determining the rate of change according to said plurality of measurements; and determining at least one characteristic of said fetal development or condition according to the rate of change.

47. The method of claim 46, wherein said rate of change is determined for a ratio of measurements of a plurality of markers.

48. The method of claim 47, wherein said plurality of markers comprise at least AFP and

49. The method of claim 48, wherein said at least one characteristic is predictive of aneuploidy.

50. The method of claim 49, wherein said aneuploidy is Down's syndrome.

51. The method of claim 46, wherein said determining said at least one characteristic further comprises determining at least one of maternal age, ethnicity or an ultrasound marker as additional data; and adjusting said determining of said at least one characteristic according to said additional data.

52. A method for evaluating the risk of fetal aneuploidy according to claim 1, the method comprising: obtaining a biological sample from a pregnant subject; determining at least two concentrations of a biomarker from said sample; evaluating a ratio comprising a numerator and a denominator wherein said numerator comprises a biomarker whose concentration is lower than the norm and wherein said denominator comprise a biomarker whose concentration is higher than the norm; and obtaining a probability of the risk from said ratio by comparing to a population distribution.

53. The method of claim 52 wherein said numerator comprises a biomarker chosen from the group consisting of alpha-fetoprotein (AFP)>unconjugated oestriol (UE3), human placental lactogen (HPL), Pregnancy-Associated Plasma Protein A (PAPP-A), estrone, estradiol, and progesterone.

54. The method of claim 52 wherein said denominator is chosen from the group consisting of human chorionic gonadotropin (hCG), free beta-hCG and inhibin-A.

55. The method of claim 52, wherein said ratio is compared to population distribution for normal and aneuploid pregnancies.

56. The method of claim 52, wherein said ratio is measured at least once during the first or second trimester of the pregnancy.

57. The method of claim 52, wherein said ratio is measured at least once during weeks 8-22 of the pregnancy.

58. The method of claim 52, further comprising measuring at least one other pregnancy related parameter,

59. The method of claim 52, further comprising determining a plurality of ratios of different markers.

60. The method of claim 59, wherein said ratio is determined for a plurality of time points, wherein a rate of change of said ratio is determined for determining said risk.

61. The method of claims 52, further comprising determining whether said ratio is outside of a false positive result area or curve.

62. The method of claims 52, wherein a distribution of said rate of change is compared for normal and aneuploid pregnancies.

63. The method of claim 52, wherein said aneuploid syndrome is Down's syndrome.

64. A method for evaluating the risk of fetal aneuploidy according to claim 28, the method comprising: obtaining at least two biological samples from a pregnant subject at least 1 week apart; determining a ratio of at least two biomarker concentrations from each of said sample; evaluating a ratio between the concentrations of each sample; determining an aneuploidy risk probability from each of said samples by comparing to a population distribution; comparing the risk probability between each of said sample to determine the rate of change of the risk probability between successive samples taken at least one week apart.

65. The method of claim 64 wherein each sample is obtained during a time period selected from the list consisting of: the first trimester of pregnancy, the second trimester of pregnancy and prior to week 23 of gestation.

66. A method for evaluating the risk of fetal aneuploidy, the method comprising: obtaining a plurality of biological samples from a pregnant subject at least one week apart; determining the concentrations of at least two biomarker from said sample; evaluating a ratio between the concentrations from each sample; evaluating the rate of change said ratio to obtain a probability of said risk from said ratio by comparing to a population, distribution.

67. The method of claim 66 wherein said plurality of biological samples are taken during the first trimester.

68. The method of claim 66 wherein a first biological sample is obtained during the first trimester and a second biological sample is obtained during the second trimester.

69. The method of claim 66 wherein said plurality of biological samples are taken during the second trimester.

70. The method of claim 66 wherein said at least two biomarkers are chosen from the group consisting of on a first concentration for fb-hCG and a second concentration chosen from the group consisting of AFP and PAPP-A.

71. The method of claim 66 wherein said ratio is chosen from the group consisting of AFP/fb-hCG and PAPP-A/fb-hCG.
